(19) United States
(12) Patent Application Publication
(10) Pub. No.: US 2025/0145588 A1
(43) Pub. Date: May 8, 2025

(54) CRYSTAL FORM OF TAVAPADON, AND PREPARATION METHOD THEREFOR AND USE THEREOF

(71) Applicant: CRYSTAL PHARMACEUTICAL (SUZHOU) CO., LTD., Suzhou, Jiangsu (CN)

(72) Inventors: Haiyan QIN, Suzhou (CN); Jiaming SHI, Suzhou (CN)

(73) Assignee: CRYSTAL PHARMACEUTICAL (SUZHOU) CO., LTD., Suzhou, Jiangsu (CN)

(21) Appl. No.: 18/832,506

(22) PCT Filed: Jan. 18, 2023
(86) PCT No.: PCT/CN2023/072961

§ 371 Date: Jul. 23, 2024

(30) Foreign Application Priority Data
Jan. 29, 2022 (CN) 202210109817.7
Jan. 29, 2022 (CN) 202210109831.7

(51) Int. Cl. C07D 401/12; A61K 31/513

(52) U.S. Cl. CPC C07D 401/12; A61K 31/513

(57) ABSTRACT

A novel crystalline form of Tavapadon (hereinafter referred to as “Compound I”) and its preparation method, pharmaceutical compositions containing this crystalline form, and its use in the preparation of dopamine D1/D5 receptor agonist drugs and in the treatment of Parkinson's disease. The crystalline form of Compound I provided by the present disclosure has one or more improved properties compared to prior arts, which solve the problems of the prior art and is of great value to the optimization and development of the drugs.

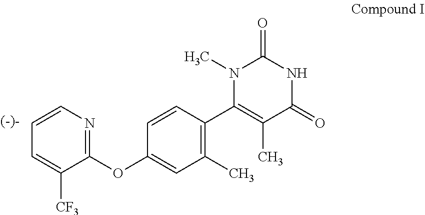 [SMILES: Cc1cc(Oc2ncccc2C(F)(F)F)ccc1-c1c(C)c(=O)[nH]c(=O)n1C]

### TECHNICAL FIELD

The present disclosure pertains to the field of chemical crystallography, particularly relates to novel crystalline forms of Tavapadon, preparation method and use thereof.

### BACKGROUND

A chronic neurodegenerative disorder, Parkinson's disease primarily results in progressive and debilitating motor symptoms, including decreased bodily movement, slowness of movement, rigidity, tremor and postural instability. On a global scale, the growth rate of disability and death attributed to Parkinson's disease is faster than any other neurological disease. Human beings still need to develop new and more effective drugs for treating Parkinson's disease.

From a pathological perspective, Parkinson's disease results from the death of dopamine-producing neurons in the brain. Dopamine is a neurotransmitter that drives motor function through a complex interaction between the striatum, the thalamus and the motor cortex. Of dopamine receptor subtypes, D1/D5 receptor subtypes are expressed in a subset of neurons whose function is to modulate signaling from the thalamus to the cortex. This pathway is known as the direct motor pathway, and is responsible for the appropriate initiation of motor activity. D2/D3 receptor subtypes, expressed by a different group of neurons, signals via the indirect motor pathway, which indirectly regulates the signaling from the thalamus to the cortex. This pathway results in inhibition of motor activity. The balance between these two groups of neurons allows for proper motor control.

Surprisingly, Tavapadon, a small molecule drug developed by Cerevel Therapeutics, has achieved positive results in the clinical stage for the treatment of early and late Parkinson's disease. It is the first oral D1/D5 receptor agonist that can improve patient movement symptoms. The chemical name of Tavapadon is (−)-1,5-dimethyl-6-(2-methyl-4-((3-(trifluoromethyl) pyridin-2-yl)oxy)phenyl)pyrimidine-2,4(1H,3H)-dione (hereinafter referred to as Compound I), and the structure is shown as the follows:

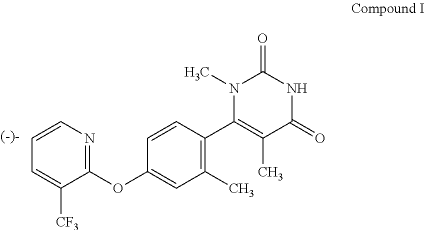 [SMILES: Cc1cc(Oc2ncccc2C(F)(F)F)ccc1-c1c(C)c(=O)[nH]c(=O)n1C]

In the development of small molecule drugs, drug polymorphism is a common phenomenon in drug development and an important factor affecting drug quality. A crystalline form is a solid formed by the three-dimensional ordered arrangement of compound molecules in a microstructure to form a lattice. Polymorphism refers to the phenomenon that a compound exists in multiple crystalline forms. Compounds may exist in one or more crystalline forms, but their existence and characteristics cannot be specifically predicted. Different crystalline forms of drug substances have different physicochemical properties, which can affect drug's in vivo dissolution and absorption and will further affect drug's clinical efficacy and safety to some extent. Especially for some poorly soluble oral solid or semi-solid dosage forms, crystalline forms are crucial to product performance. Moreover, the physiochemical properties of a crystalline form are essential to a production process.

Therefore, to obtain a crystalline form of Compound I that is beneficial for production or formulation, it is necessary to conduct a comprehensive investigation of its crystallization behavior to obtain a crystalline form that meets the pharmaceutical needs of Compound I.

Through extensive research, the inventors found that only one prior art, WO2014207601A1, is related to Compound I. However, this prior art only applies to protect the compound structure of Compound I, and only its structure, preparation method and use are disclosed in the specification, also no polymorphism studies were conducted. More importantly, Example 7 of the prior art WO2014207601A1 only discloses a solid of Compound I. The inventors of the present disclosure repeated the preparation method, and found through XRPD testing that the obtained solid differed from the crystalline forms of Compound I provided by the present disclosure. Furthermore, after a careful study of the prior art solid by the inventors of the present disclosure, it is found that the solid has poor stability, and is prone to crystal transformation, making it unsuitable for medicinal use.

To overcome the disadvantages of prior arts, a novel crystalline form meeting the medicinal standard is still needed for the development of drugs containing Compound I. Through extensive research, the inventors found that Compound I is very easy to form a solvate. The inventors obtained 11 solvates of different solvates of Compound I, including 1,4-dioxane solvate, toluene solvate, acetic acid solvate, hexafluoroisopropanol solvate, 2-Methyltetrahydrofuran solvate, tetrahydrofuran solvate, dimethyl carbonate solvate, diethyl carbonate solvate, chlorobenzene solvate, anisole solvate, benzyl alcohol solvate. However, due to the high content of organic solvents, these solvates are unsuitable for medicinal use. Therefore, it is challenging to obtain a new polymorph that meets medicinal standards. After extensive creative work,
the inventors unexpectedly found crystalline forms of Compound I of the present disclosure, which have advantages in at least one aspect of solubility, hygroscopicity, purification ability, stability, adhesiveness, compressibility, flowability, in vitro and in vivo dissolution, and bioavailability, etc. In particular, the crystalline forms have good stability, low hygroscopicity, no residual solvent, high purity, and excellent purification ability, solving the problems in the prior art, and having significant importance for the development of drugs containing Compound I.

### SUMMARY

The present disclosure is to provide novel crystalline forms of Compound I, preparation method, use and pharmaceutical compositions comprising crystalline forms.

According to the objective of the present disclosure, crystalline form CSI of Compound I is provided (hereinafter referred to as Form CSI).

In one aspect provided herein, the X-ray powder diffraction pattern of Form CSI comprises one or two or three characteristic peaks at 2theta values of 15.7°±0.2°, 20.0°±0.2° and 25.0°±0.2° using CuKα radiation. Preferably, the X-ray powder diffraction pattern of Form CSI comprises characteristic peaks at 2theta values of 15.7°±0.2°, 20.0°±0.2° and 25.0°±0.2° using CuKα radiation.

Furthermore, the X-ray powder diffraction pattern of Form CSI comprises one or two or three characteristic peaks at 2theta values of 17.9°±0.2°, 21.2°±0.2° and 24.0°±0.2° using CuKα radiation. Preferably, the X-ray powder diffraction pattern of Form CSI comprises characteristic peaks at 2theta values of 17.9°±0.2°, 21.2°±0.2° and 24.0°±0.2° using CuKα radiation.

Furthermore, the X-ray powder diffraction pattern of Form CSI comprises one or two or three characteristic peaks at 2theta values of 8.4°±0.2°, 16.4°±0.2° and 24.5°±0.2° using CuKα radiation. Preferably, the X-ray powder diffraction pattern of Form CSI comprises characteristic peaks at 2theta values of 8.4°±0.2°, 16.4°±0.2° and 24.5°±0.2° using CuKα radiation.

In another aspect provided herein, the X-ray powder diffraction pattern of Form CSI comprises one or two or three or four or five or six or seven or eight or nine or ten or eleven or twelve or thirteen or fourteen or fifteen or sixteen characteristic peaks at 2theta values of 15.7°±0.2°, 20.0°±0.2°, 25.0°±0.2°, 17.9°±0.2°, 24.0°±0.2°, 21.2°±0.2°, 8.4°±0.2°, 16.4°±0.2°, 24.5°±0.2°, 9.3°±0.2°, 11.7°±0.2°, 13.8°±0.2°, 14.9°±0.2°, 18.5°±0.2°, 29.1°±0.2° and 32.7°±0.2° using CuKα radiation.

Without any limitation being implied, the X-ray powder diffraction pattern of Form CSI is substantially as shown in FIG. 1 using CuKα radiation.

Without any limitation being implied, Form CSI is an anhydrate.

According to the objective of the present disclosure, a process for preparing Form CSI is also provided. The process comprises: adding Compound I into an alcohol or a ketone, stirring, separating to obtain Form CSI.

According to the objective of the present disclosure, crystalline Form CSII of Compound I is provided (hereinafter referred to as Form CSII).

In one aspect provided herein, the X-ray powder diffraction pattern of Form CSII comprises one or two or three characteristic peaks at 2theta values of 7.8°±0.2°, 15.1°±0.2° and 17.0°±0.2° using CuKα radiation. Preferably, the X-ray powder diffraction pattern of Form CSII comprises characteristic peaks at 2theta values of 7.8°±0.2°, 15.1°±0.2° and 17.0°±0.2° using CuKα radiation.

Furthermore, the X-ray powder diffraction pattern of Form CSII comprises one or two or three characteristic peaks at 2theta values of 18.7°±0.2°, 23.2°±0.2° and 26.0°±0.2° using CuKα radiation. Preferably, the X-ray powder diffraction pattern of Form CSII comprises characteristic peaks at 2theta values of 18.7°±0.2°, 23.2°±0.2° and 26.0°±0.2° using CuKα radiation.

Furthermore, the X-ray powder diffraction pattern of Form CSII comprises one or two characteristic peaks at 2theta values of 23.8°±0.2° and 24.4°±0.2° using CuKα radiation. Preferably, the X-ray powder diffraction pattern of Form CSII comprises characteristic peaks at 2theta values of 23.8°±0.2° and 24.4°±0.2° using CuKα radiation.

In another aspect provided herein, the X-ray powder diffraction pattern of Form CSII comprises three or four or five or six or seven or eight or nine or ten or eleven or twelve or thirteen characteristic peaks at 2theta values of 7.8°±0.2°, 15.1°±0.2°, 17.0°±0.2°, 18.7°±0.2°, 23.2°±0.2°, 26.0°±0.2°, 23.8°±0.2°, 24.4°±0.2°, 9.3°±0.2°, 13.8°±0.2°, 21.4°±0.2°, 31.8°±0.2° and 32.8°±0.2° using CuKα radiation.

In another aspect provided herein, the X-ray powder diffraction pattern of Form CSII comprises three or four or five or six or seven or eight or nine or ten or eleven or twelve or thirteen characteristic peaks at 2theta values of 7.8°±0.2°, 15.1°±0.2°, 17.0°±0.2°, 18.7°±0.2°, 23.2°±0.2°, 26.0°±0.2°, 23.8°±0.2°, 24.4°±0.2°, 9.3°±0.2°, 13.8°±0.2°, 21.4°±0.2°, 31.8°±0.2° and 32.8°±0.2° using CuKα radiation, and comprises no characteristic peaks at 2theta values of 19.2°-20.5° or 24.8°-25.6°.

In another aspect provided herein, the X-ray powder diffraction pattern of Form CSII comprises three or four or five or six or seven or eight or nine or ten or eleven or twelve or thirteen characteristic peaks at 2theta values of 7.8°±0.2°, 15.1°±0.2°, 17.0°±0.2°, 18.7°±0.2°, 23.2°±0.2°, 26.0°±0.2°, 23.8°±0.2°, 24.4°±0.2°, 9.3°±0.2°, 13.8°±0.2°, 21.4°±0.2°, 31.8°±0.2° and 32.8°±0.2° using CuKα radiation, and comprises no characteristic peaks at 2theta values of 19.2°-20.5° and 24.8°-25.6°.

Without any limitation being implied, the X-ray powder diffraction pattern of Form CSII is substantially as shown in FIG. 4 using CuKα radiation.

Without any limitation being implied, Form CSII is an anhydrate.

According to the objective of the present disclosure, a process for preparing Form CSII is also provided. The process comprises: heating Compound I solid, cooling to room temperature to obtain Form CSII.

Furthermore, temperature of said heating is preferably 233-238° C., more preferably 235° C.

According to the objective of the present disclosure, a pharmaceutical composition is provided, said pharmaceutical composition comprises a therapeutically effective amount of Form CSI, Form CSII or any mixture of the two crystalline forms, along with pharmaceutically acceptable excipients.

Furthermore, Form CSI, Form CSII or any mixture of the two crystalline forms can be used for preparing dopamine D1/D5 receptor agonist drugs.

Furthermore, Form CSI, Form CSII or any mixture of the two crystalline forms can be used for preparing drugs treating Parkinson's disease.

### Beneficial Effects

Form CSI of the present disclosure has the following beneficial effects:
(1) Compared with the prior art solid, From CSI drug substance of the present disclosure has better physicochemical stability.

The prior art solid undergoes crystalline transformation and exhibits poor stability after being stored at 40° C./75% RH for 10 days. In contrast, Form CSI remains unchanged after being stored for at least 6 months at 40° C./75% RH, and at least 6 months when stored at 60° C./75% RH with sealed package. During storage, its purity remains substantially unchanged. This indicates that Form CSI exhibits better stability under accelerated and more stress conditions compared to the prior art solid.

Drug substance and drug product would go through high temperature and high humidity conditions caused by different season, regional climate and environment during storage, transportation, and manufacturing processes. Therefore, good stability under accelerated and stress conditions is of great importance to the drug development. Form CSI drug substance has good stability under stress condition both itself and in drug product, which is beneficial to avoid the impact on drug quality due to crystal transformation or decrease in purity during drug storage.
(2) Form CSI exhibits good humidity stability. After undergoing one cycle of 0% RH-95% RH-0% RH, Form CSI remains unchanged.
(3) Form CSI has good physical stability under mechanical force. The crystalline state of Form CSI doesn't change after grinding. Grinding and pulverization are often required in the drug manufacturing process. Good physical stability of the drug substance can reduce the risk of crystallinity decrease and crystal transformation during the drug production process.

Good physical and chemical stability of drug substance ensure that no crystal transformation or impurities is generated during production and storage. Form CSI has good physical and chemical stability, ensuring consistent and controllable quality of the drug substance and drug product, minimizing quality change, bioavailability change and toxicity due to crystal transformation or impurity generation.
(4) Form CSI of the present disclosure has good purification effect. The purity is significantly increased after the starting material is converted into Form CSI. In a specific embodiment of the present disclosure, the purity of the starting material used is 97.78%. The purity of Form CSI made from the starting material is 99.85%. The purity is increased over 2%. Meanwhile, the number of impurities decreased by 5, and the maximum single impurity decreased from 1.84% to 0.06%.

Chemical purity is of great significance for ensuring drug efficacy, safety and preventing the occurrence of adverse effects. Drug regulations have strict requirements on impurity content. As Form CSI of the present disclosure has good purification ability and is excellent in removing impurities in the crystallization process. Thus drug substances with high purity can be obtained through crystallization, which effectively overcome the disadvantages of poor stability, poor efficacy and high toxicity caused by the low purity drug substances.
(5) Form CSI of the present disclosure has no residual solvent. Residual solvents can affect drug safety, quality and stability, potentially leading to crystalline transformation or impurity formation during drug's production and storage, which can affect drug bioavailability and toxicity. The absence of residual solvents in Form CSI effectively addresses issues related to low drug stability, poor efficacy, and high toxicity caused by low drug purity or high solvent residues.
(6) Form CSI of the present disclosure has almost no hygroscopicity. The test results show that the weight gain of Form CSI at 80% RH is only 0.05%.

In one aspect, high hygroscopicity tends to cause chemical degradation and polymorph transformation, which affects the physicochemical stability of the drug substance. In addition, high hygroscopicity will reduce the flowability of the drug substance, thereby affecting the processing of the drug substance. In another aspect, drug substance with high hygroscopicity requires low humidity environment during production and storage, which puts strict requirements on production and imposes higher costs. More importantly, high hygroscopicity is likely to cause variation in the content of active pharmaceutical ingredients in the drug product, thus affecting drug product quality.

Form CSI provided by the present disclosure with low hygroscopicity is not demanding on the production and storage conditions, which reduces the cost of production, storage and quality control, and has strong economic value.

Form CSII of the present disclosure has the following beneficial effects:
(1) Compared with the prior art solid, From CSII drug substance of the present disclosure has better physicochemical stability.

The prior art solid undergoes crystalline transformation and exhibits poor stability after being stored at 40° C./75% RH for 10 days. In contrast, Form CSII remains unchanged after being stored for at least one month at 40° C./75% RH with sealed package. This indicates that Form CSII exhibits better stability under accelerated conditions compared to the prior art solid.

Drug substance and drug product would go through high temperature and high humidity conditions caused by different season, regional climate and environment during storage, transportation, and manufacturing processes. Therefore, good stability under accelerated and stress conditions is of great importance to the drug development. Form CSII drug substance has good stability under stress condition both itself and in drug product, which is beneficial to avoid the impact on drug quality due to crystal transformation or decrease in purity during drug storage.
(2) Form CSII exhibits good humidity stability. After undergoing one cycle of 0% RH-95% RH-0% RH, Form CSII remains unchanged.
(3) Form CSII has good physical stability under mechanical force. The crystalline state of Form CSII doesn't change after grinding. Grinding and pulverization are often required in the drug manufacturing process. Good physical stability of the drug substance can reduce the risk of crystallinity decrease and crystal transformation during the drug manufacturing process.

Good physical and chemical stability of drug substance ensure that no crystal transformation or impurities is generated during production and storage. Form CSII has good physical and chemical stability, ensuring consistent and controllable quality of the drug substance and drug product, minimizing quality change, bioavailability change and toxicity due to crystal transformation or impurity generation.
(4) Form CSII of the present disclosure has no residual solvent. Residual solvents can affect drug safety, quality and stability, potentially leading to crystalline transformation or impurity formation during drug's production and storage, which can affect drug bioavailability and toxicity. The absence of residual solvents in Form CSII effectively addresses issues related to low drug stability, poor efficacy, and high toxicity caused by low drug purity or high solvent residues.
(5) Form CSII of the present disclosure has almost no hygroscopicity. The test results show that the weight gain of Form CSII at 80% RH is only 0.19%.

In one aspect, high hygroscopicity tends to cause chemical degradation and polymorph transformation, which affects the physicochemical stability of the drug substance. In addition, high hygroscopicity will reduce the flowability of the drug substance, thereby affecting the processing of the drug substance. In another aspect, drug substance with high hygroscopicity requires low humidity environment during production and storage, which puts strict requirements on production and imposes higher costs. More importantly, high hygroscopicity is likely to cause variation in the content of active pharmaceutical ingredients in the drug product, thus affecting drug product quality.

Form CSII provided by the present disclosure with low hygroscopicity is not demanding on the production and storage conditions, which reduces the cost of production, storage and quality control, and has strong economic value.

### BRIEF DESCRIPTION OF THE DRAWINGS

FIG. 1 shows an XRPD pattern of Form CSI;

FIG. 2 shows a TGA curve of Form CSI;

FIG. 3 shows a DSC curve of Form CSI;

FIG. 4 shows an XRPD pattern of Form CSII;

FIG. 5 shows a TGA curve of Form CSII;

FIG. 6 shows a DSC curve of Form CSII;

FIG. 7 shows an XRPD pattern overlay of Form CSI and the prior art solid (top: Form CSI, bottom: the prior art solid);

FIG. 8 shows an XRPD pattern overlay of Form CSII and the prior art solid (top: Form CSII, bottom: the prior art solid);

FIG. 9 shows an XRPD pattern overlay of prior art solid before and after being stored under 40° C./75% RH (sealed with desiccant) for 10 days (top: after storage, bottom: before storage);

FIG. 10 shows an XRPD pattern overlay of Form CSI of the present disclosure before and after storage under different conditions (Form top to bottom: initial, 25° C./60% RH open for 6 months, 25° C./60% RH sealed for 6 months (with desiccants), 40° C./75% RH open for 6 months, 40° C./75% RH sealed for 6 months (with desiccants), 60° C./75% RH sealed for 6 months (with desiccants));

FIG. 11 shows a DVS adsorption curve of Form CSI;

FIG. 12 shows an XRPD pattern overlay of Form CSI before and after DVS test (top: before test, bottom: after test);

FIG. 13 shows an XRPD pattern overlay of Form CSI before and after grinding (top: before grinding, bottom: after grinding);

FIG. 14 shows a DVS adsorption curve of Form CSII;

FIG. 15 shows an XRPD pattern overlay of Form CSII before and after DVS test (top: before test, bottom: after test);

FIG. 16 shows an XRPD pattern overlay of Form CSII before and after grinding (top: before grinding, bottom: after grinding).

### DETAILED DESCRIPTION

The present disclosure is further illustrated by the following examples which describe the preparation and use of the crystalline forms of the present disclosure in detail. It is obvious to those skilled in the art that changes in the materials and methods can be accomplished without departing from the scope of the present disclosure.

The abbreviations used in the present disclosure are explained as follows:
XRPD: X-ray Powder Diffraction
TGA: Thermo Gravimetric Analysis
DSC: Differential Scanning calorimetry
¹H NMR: Proton Nuclear Magnetic Resonance
DVS: Dynamic Vapor Sorption
HPLC: High Performance Liquid Chromatography
RH: Relative Humidity

### Instruments and Methods Used for Data Collection:

XRPD patterns in the present disclosure were acquired by a Bruker X-ray powder diffractometer. The parameters of the X-ray powder diffraction method of the present disclosure are as follows:
X-Ray source: Cu, Kα
Kα1 (Å): 1.54060; Kα2 (Å): 1.54439
Kα2/Kα1 intensity ratio: 0.50

TGA data in the present disclosure were acquired by a TA Q500. The parameters of the TGA method of the present disclosure are as follows:
Heating rate: 10° C./min
Purge gas: N₂

DSC data in the present disclosure were acquired by a TA Q2000. The parameters of the DSC method of the present disclosure are as follows:
Heating rate: 10° C./min
Purge gas: N₂

DVS data in the present disclosure were measured via an SMS (Surface Measurement Systems Ltd.) intrinsic DVS instrument. The instrument control software is DVS-Intrinsic control software. Typical Parameters for DVS test are as follows:
Temperature: 25° C.
Gas and flow rate: N₂, 200 mL/minRH range: 0% RH to 95% RH

¹H NMR data were collected from a Bruker Avance II DMX 400M HZ NMR spectrometer. 1-5 mg of sample was weighed and dissolved with 0.5 mL of deuterated chloroform to obtain a solution with a concentration of 2-10 mg/mL.

The parameters for related substance detection in the present disclosure are shown in Table 1.

TABLE 1

| HPLC | Agilent 1260 with DAD/VWD detector | |
|---|---|---|
| Column | Waters XBridge C18, 4.6 mm*150 mm, 3.5 μm | |
| Ghost-Buster column | Welch Ghost-Buster, 4.6*50 mm | |
| Mobile phase | A: 0.1% phosphoric acid aqueous solution | |
| | B: methanol:acetonitrile = 700:300 | |
| | Time (min) | % B |
| Gradient | 0.0 | 10 |
| | 20.0 | 80 |
| | 30.0 | 80 |
| | 31.0 | 10 |
| | 40.0 | 10 |

| | |
|---|---|
| Flow rate | 1.0 mL/min |
| Injection volume | 5 μL |
| Detector wavelength | 274 nm |
| Column temperature | 35° C. |
| Sample plate temperature | Room temperature |
| Diluent | Methanol |

In the present disclosure, said “stirring” is accomplished by using a conventional method in the field such as magnetic stirring or mechanical stirring and the stirring speed is 50 to 1800 r/min. Preferably the magnetic stirring speed is 300 to 900 r/min, and mechanical stirring speed is 100 to 300 r/min.

Said “separation” is accomplished by using a conventional method in the field such as centrifugation or filtration. The operation of “centrifugation” is as follows: the sample to be separated is placed into the centrifuge tube, and then centrifuged at a rate of 10000 r/min until the solid all sink to the bottom of the tube.

Said “drying” is accomplished by using a conventional method in the field such as vacuum drying, blast drying or free-air drying. The drying temperature can be room temperature or higher. Preferably the drying temperature is from room temperature to about 60° C., or to 50° C., or to 40° C. The drying time can be 2 to 48 hours, or overnight. Drying is accomplished in a fume hood, forced air convection oven or vacuum oven.

Said “room temperature” is not a specific temperature, but a temperature range of 10-30° C.

Said “characteristic peak” refers to a representative diffraction peak used to distinguish crystals, which usually can have a deviation of +0.2° using CuKα radiation.

Said “anhydrate” refers to a solid substance that does not contain crystalline water or solvents.

In the present disclosure, “crystal” or “crystalline form” refers to the crystal or the crystalline form being identified by the X-ray diffraction pattern shown herein. The XRPD diffraction data of crystals have fingerprint characteristics. In this field, different crystals are identified based on the XRPD diffraction data. Those skilled in the art will select several representative peaks in the XRPD pattern as characteristic peaks to characterize crystals. When selecting characteristic peaks, the peak position, peak intensity, and peak shape will be comprehensively considered. However, those skilled in the art are able to understand that the X-ray diffraction pattern typically varies with the instrument conditions, the sample preparation and the purity of samples. The peak intensity in the X-ray diffraction pattern typically varies with the experimental conditions. In fact, the relative intensity of diffraction peaks in the XRPD pattern is related to the preferential orientation of crystals, and the diffraction peak intensities provided in this disclosure are illustrative rather than for absolute comparison. Therefore, when identifying whether the crystals are the same, the matching of peak positions is the first priority.

It will be understood by those skilled in the art that a crystalline form of the present disclosure is not necessarily to have exactly the same X-ray diffraction pattern of the example shown herein. Any crystalline forms whose X-ray diffraction patterns have the same or similar characteristic peaks should be within the scope of the present disclosure. Those skilled in the art can compare the patterns shown in the present disclosure with that of an unknown crystalline form in order to identify whether these two groups of patterns reflect the same or different crystalline forms.

In some embodiments, Form CSI and Form CSII of the present disclosure are pure and substantially free of any other crystalline forms. In the present disclosure, the term “substantially free” when used to describe a novel crystalline form, it means that the content of other crystalline forms in the novel crystalline form is less than 20% (w/w), specifically less than 10% (w/w), more specifically less than 5% (w/w) and furthermore specifically less than 1% (w/w).

In the present disclosure, the term “about” when referring to a measurable value such as weight, time, temperature, and the like, is meant to encompass variations of ±10%, ±5%, ±1%, ±0.5%, or even ±0.1% of the specified amount.

Unless otherwise specified, the following examples were conducted at room temperature.

According to the present disclosure, Compound I used as raw materials include, but are not limited to solid (crystalline and amorphous), oil, liquid form or solution. Preferably, Compound I used as the raw material is a solid.

Raw materials of Compound I used in the following examples were prepared by known methods in the prior art, for example, the method disclosed in WO2014207601A1.

### Example 1: Preparation of Form CSI

As shown in Table 2, a certain mass of Compound I solids were weighed into glass vials, followed by addition of a certain volume of solvents to form a suspension. After stirring at room temperature for 2 days, the solids were separated. The obtained solids were marked as sample 1 and sample 2 as shown in Table 2. After XRPD testing, sample 1 and sample 2 are Form CSI of the present disclosure.

TABLE 2

| | Weight of | | | |
|---|---|---|---|---|
| Sample | Compound I (mg) | Solvent | Volume (mL) | Solid form |
| 1 | 10.2 | Methanol | 0.2 | Form CSI |
| 2 | 11.3 | 2-Butanone | 0.2 | Form CSI |

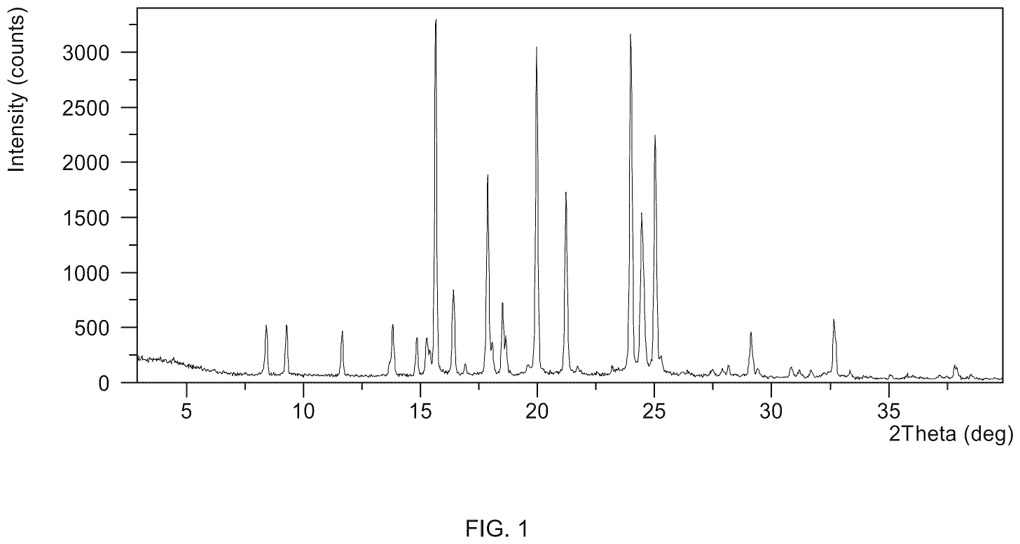

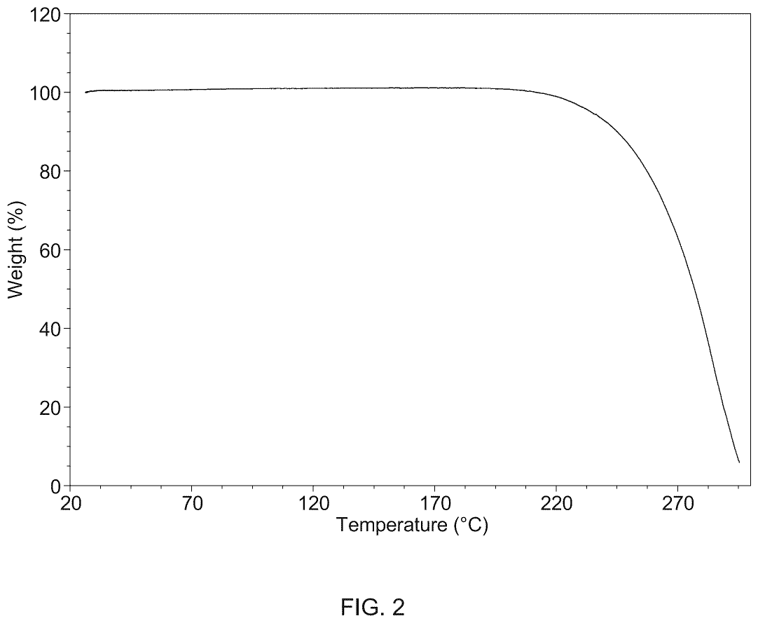

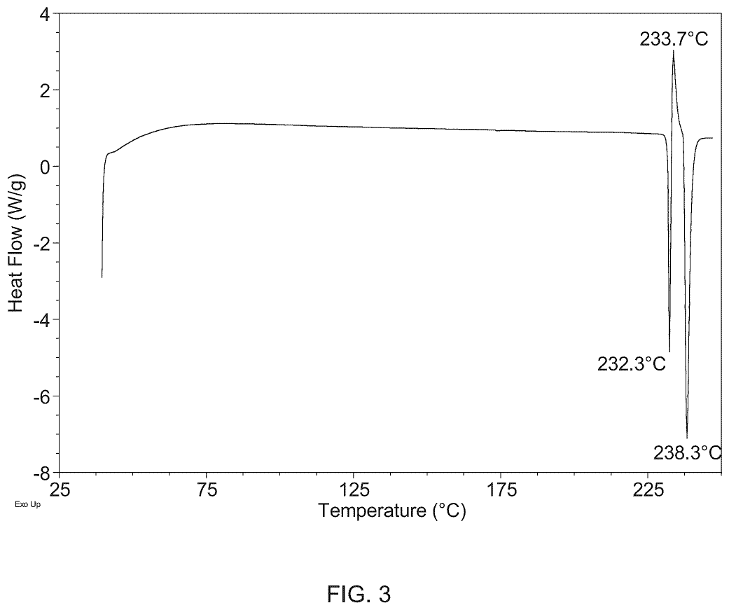

The dry solid was obtained by vacuum drying sample 1 at 50° C. for about 2 hours, and the dry solid was confirmed as Form CSI by XRPD. The XRPD pattern is shown in FIG. 1, and the XRPD data are listed in Table 3. The TGA curve shows Form CSI has almost no weight loss when heating to 180° C., which is substantially as shown in FIG. 2. Form CSI is an anhydrate. The DSC curve of Form CSI is substantially as shown in FIG. 3. The first endothermic peak is at around 232° C. (peak temperature), the first exothermic peak is at around 234° C. (peak temperature), and the second endothermic peak is at around 238° C. (peak temperature).

The ¹H NMR data of Form CSI are as follows: ¹H NMR (400 MHZ, Chloroform-d) δ 8.36-8.31 (m, 1H), 8.28 (s, 1H), 8.04 (dd, J=7.8, 1.9 Hz, 1H), 7.21-7.11 (m, 4H), 3.05 (s, 3H), 2.20 (s, 3H), 1.68 (s, 3H), of which the signal at δ 8.28 ppm belongs to an exchangeable hydrogen. NMR data show that there is no residual solvent in Form CSI.

TABLE 3

| 2θ (°) | d spacing (Å) | Relative intensity (%) |
|---|---|---|
| 8.42 | 10.50 | 13.70 |
| 9.28 | 9.53 | 14.03 |
| 11.66 | 7.59 | 12.34 |
| 13.82 | 6.41 | 14.46 |
| 14.85 | 5.97 | 10.82 |
| 15.28 | 5.80 | 10.69 |
| 15.65 | 5.66 | 100.00 |
| 16.41 | 5.40 | 24.14 |
| 16.91 | 5.24 | 3.48 |
| 17.88 | 4.96 | 55.85 |
| 18.09 | 4.90 | 8.70 |
| 18.51 | 4.79 | 20.70 |
| 18.65 | 4.76 | 11.01 |
| 19.62 | 4.53 | 3.25 |
| 19.97 | 4.45 | 92.15 |
| 21.22 | 4.19 | 50.86 |
| 21.72 | 4.09 | 2.91 |
| 23.20 | 3.83 | 2.70 |
| 23.99 | 3.71 | 95.12 |
| 24.45 | 3.64 | 45.34 |
| 25.03 | 3.56 | 66.53 |
| 26.32 | 3.39 | 1.19 |
| 27.47 | 3.25 | 2.27 |
| 27.91 | 3.20 | 2.38 |
| 28.15 | 3.17 | 3.49 |
| 29.12 | 3.07 | 12.61 |
| 30.84 | 2.90 | 3.14 |
| 31.18 | 2.87 | 2.02 |
| 31.69 | 2.82 | 2.03 |
| 32.67 | 2.74 | 16.54 |
| 33.35 | 2.69 | 1.73 |
| 35.09 | 2.56 | 0.94 |
| 37.20 | 2.42 | 0.70 |
| 37.82 | 2.38 | 3.91 |
| 38.54 | 2.34 | 1.07 |

### Example 2: Preparation of Form CSII

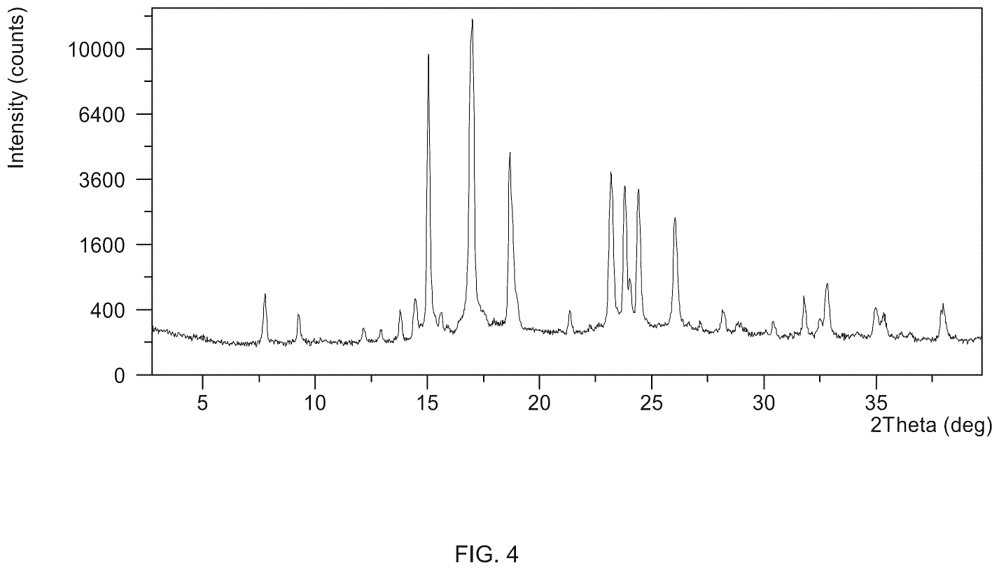

5.98 mg of Compound I solid was heated to 235° C. at 10° C./min and kept at 235° C. for 1 min, and then cooling to room temperature at 10° C./min under nitrogen protection, Form CSII was obtained. The XRPD pattern is substantially as shown in FIG. 4, and the XRPD data are listed in Table 4.

The ¹H NMR data of Form CSII are as follows: ¹H NMR (400 MHZ, Chloroform-d) δ 8.76 (s, 1H), 8.33 (dd, J=5.1, 1.8 Hz, 1H), 8.04 (dd, J=7.7, 1.8 Hz, 1H), 7.22-7.10 (m, 4H), 3.05 (s, 3H), 2.20 (s, 3H), 1.68 (s, 3H), of which the signal at & 8.76 ppm belongs to an exchangeable hydrogen. NMR data show that there is no residual solvent in Form CSII.

TABLE 4

| 2θ (°) | d spacing (Å) | Relative intensity (%) |
|---|---|---|
| 7.77 | 11.38 | 4.56 |
| 9.27 | 9.54 | 2.11 |
| 12.16 | 7.28 | 0.96 |
| 12.93 | 6.85 | 0.82 |
| 13.81 | 6.41 | 2.40 |
| 14.46 | 6.12 | 3.86 |
| 15.05 | 5.89 | 82.06 |
| 15.61 | 5.68 | 2.25 |
| 17.00 | 5.22 | 100.00 |
| 18.68 | 4.75 | 38.58 |
| 21.36 | 4.16 | 2.40 |
| 23.19 | 3.84 | 31.64 |
| 23.81 | 3.74 | 27.56 |
| 24.03 | 3.70 | 6.54 |
| 24.41 | 3.65 | 26.58 |
| 26.04 | 3.42 | 18.87 |
| 27.16 | 3.28 | 1.33 |
| 28.20 | 3.16 | 2.25 |
| 28.88 | 3.09 | 1.15 |
| 30.41 | 2.94 | 1.35 |
| 31.81 | 2.81 | 3.41 |
| 32.49 | 2.76 | 1.57 |
| 32.82 | 2.73 | 5.72 |
| 34.99 | 2.56 | 2.60 |
| 35.33 | 2.54 | 1.82 |
| 36.50 | 2.46 | 0.42 |
| 37.96 | 2.37 | 2.54 |

### Example 3: TGA and DSC of Form CSII

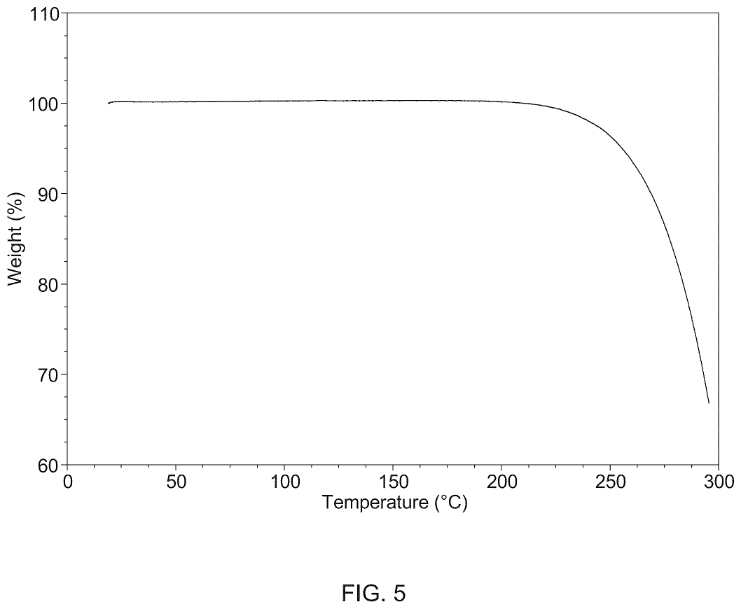

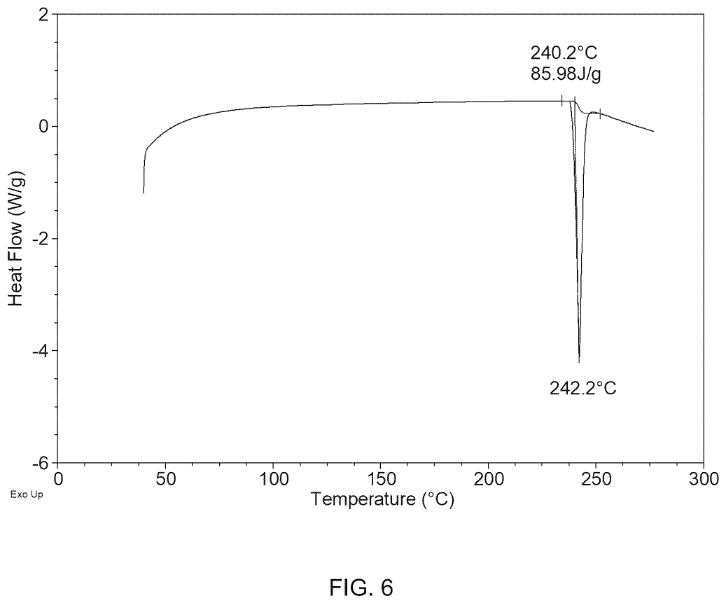

A small amount of Form CSII solid was used for TGA and DSC tests. The TGA curve shows Form CSII has almost no weight loss when heating to 200° C., which is substantially as shown in FIG. 5. The DSC curve of Form CSII is substantially as shown in FIG. 6, the first endothermic peak at around 240° C. (onset temperature) corresponds to the melting point of Form CSII.

### Example 4: Repeating the Preparation Method of Compound I Disclosed in the Prior Art

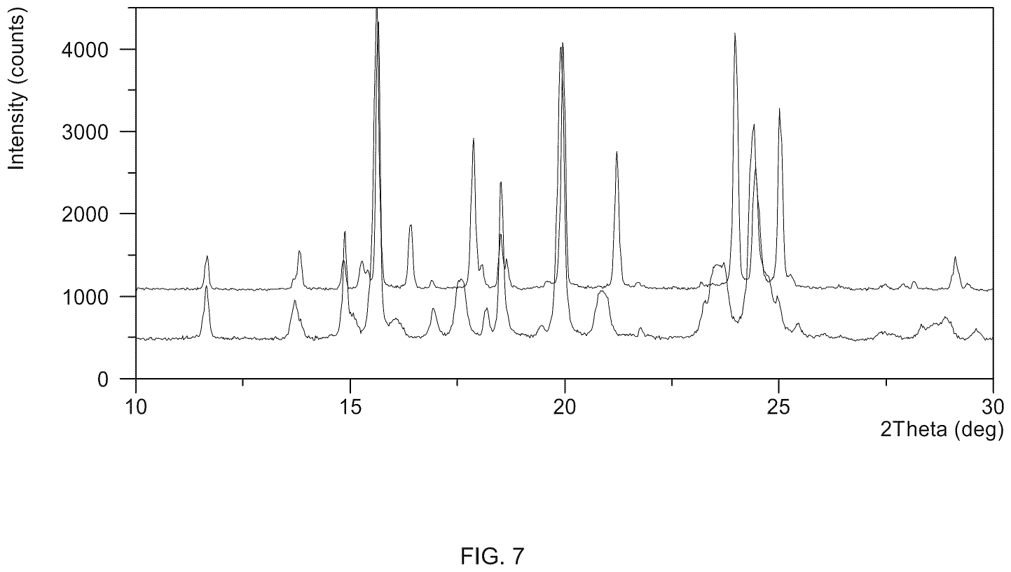

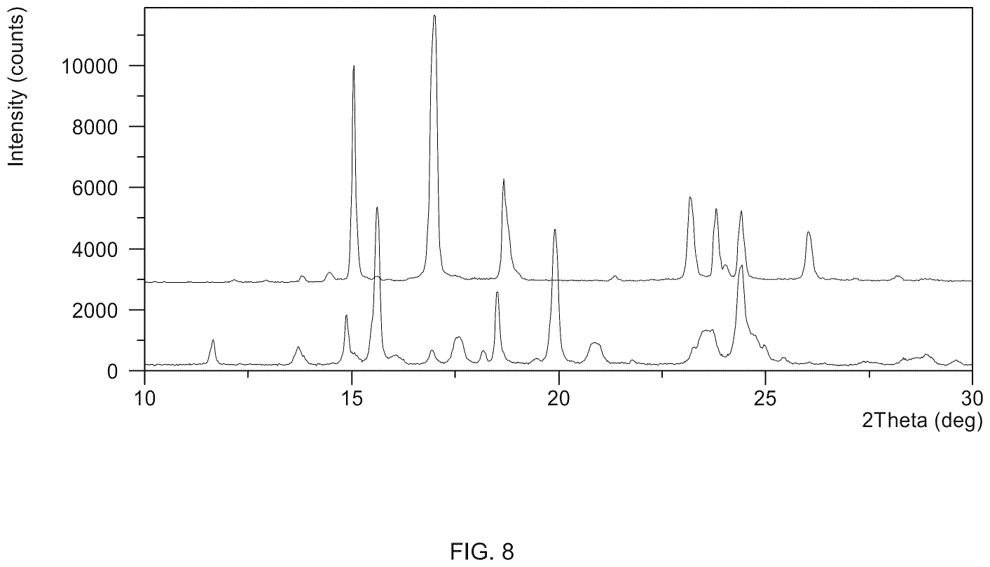

The prior art solid was obtained by repeating the preparation method of Compound I disclosed in WO2014207601A1 Example 7. The XRPD overlay of the prior art solid and Form CSI is shown in FIG. 7, and the XRPD overlay of the prior art solid and Form CSII is shown in FIG. 8. The overlays show that the XRPD pattern of the prior art solid is different from that of Form CSI and Form CSII. Specifically, the 20 values of the diffraction peaks of the prior art solid have significantly different with that of Form CSI and Form CSII in the range of 15-25°. Form CSI has diffraction peaks at 16.4°, 17.9°, 21.2° and 24.0°, while the prior art solid has no diffraction peaks at these four positions. Form CSII has diffraction peaks at 15.1°, 18.7° and 26.0°, while the prior art solid has no diffraction peaks at these three positions. Therefore, the prior art solid is different from Form CSI and Form CSII.

### Example 5: Stability of the Prior Art Solid

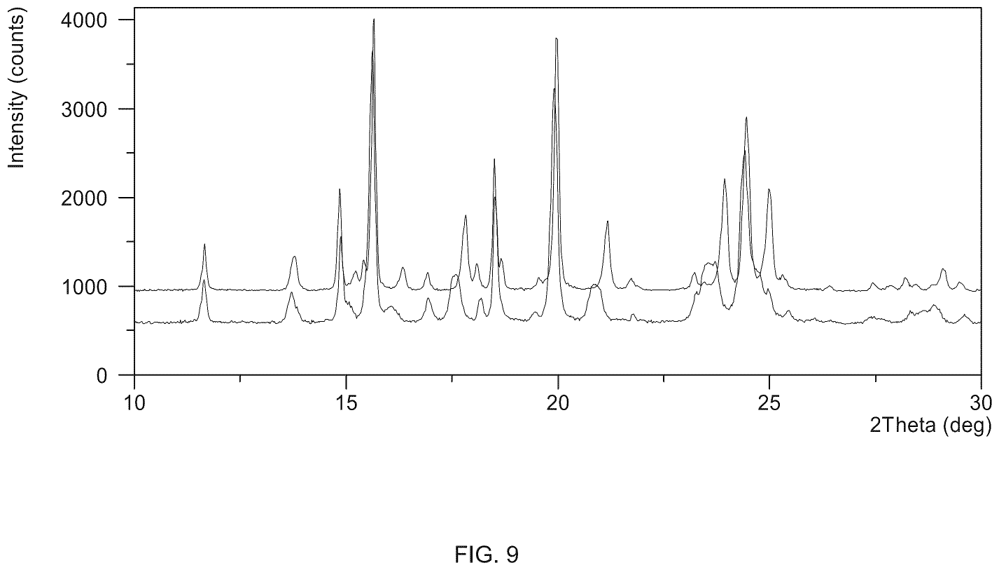

A certain amount of the prior art solid obtained in Example 4 was packaged with sealed container (with desiccant inside), and stored under 40° C./75% RH. The solid form before and after storage was confirmed by XRPD. The XRPD overlay is shown in FIG. 9. The result shows that the prior art solid undergoes crystal transformation after being stored at 40° C./75% RH for 10 days.

### Example 6: Stability of Form CSI

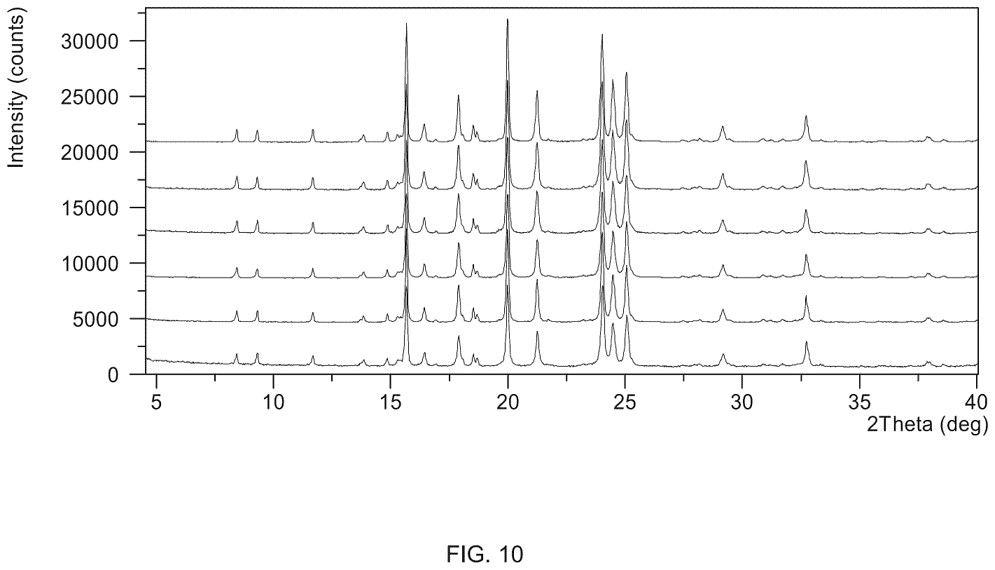

A certain amount of Form CSI was packaged with corresponding package conditions, and then stored under 25° C./60% RH, 40° C./75% RH and 60° C./75% RH. Crystalline form and chemical purity were checked by XRPD and HPLC, respectively. The results are shown in Table 5, and the XRPD overlay is shown in FIG. 10. Form CSI kept stable for at least 6 months when stored at 25° C./60% RH open and sealed (with desiccants) package, 40° C./75% RH open and sealed (with desiccants) package, and 60° C./75% RH sealed (with desiccants) package. The results show that the Form CSI has good stability and is more stable than the prior art solid.

TABLE 5

| Initial | Storage | Package | Storage | | |
|---|---|---|---|---|---|
| solid form | condition | condition | time | Solid form | Purity |
| Form CSI | Initial | — | — | Form CSI | 99.85% |
| | 25° C./ | Sealed (with | 6 months | Form CSI | 99.85% |
| | 60% RH | desiccants) | | | |
| | | Open | 6 months | Form CSI | 99.85% |
| | 40° C./ | Sealed (with | 6 months | Form CSI | 99.85% |
| | 75% RH | desiccants) | | | |
| | | Open | 6 months | Form CSI | 99.85% |
| | 60° C./ | Sealed (with | 6 months | Form CSI | 99.85% |
| | 75% RH | desiccants) | | | |

### Example 7 Purification Ability of Form CSI

HPLC was applied to test the chemical purity of raw material and Form CSI. The results shown in Table 6 indicate that Form CSI has significant purification effect.

TABLE 6

| | | | Maximum | |
|---|---|---|---|---|
| | Chemical | Purity | single | Number of |
| Solid form | purity | increment | impurity | impurities |
| Starting material | 97.78% | — | 1.84% | 9 |
| Form CSI | 99.85% | 2.07% | 0.06% | 4 |

### Example 8 Hygroscopicity of Form CSI

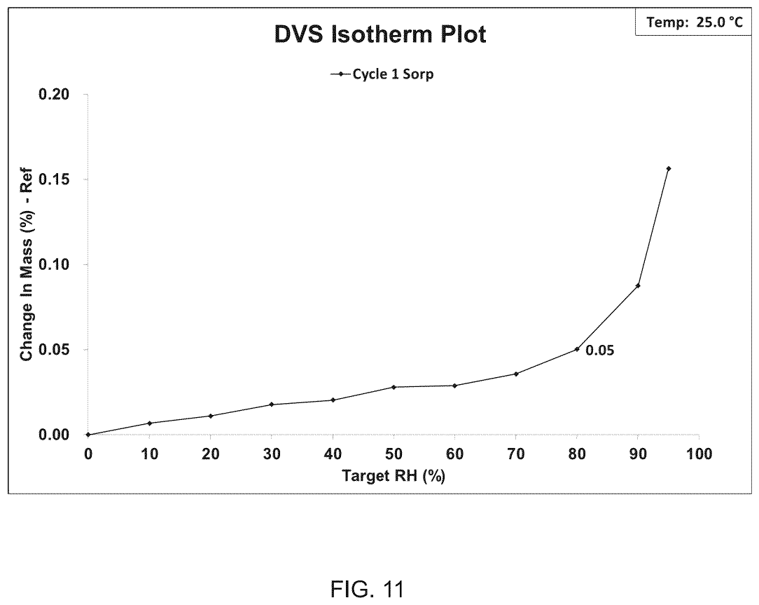

The hygroscopicity of Form CSI was determined by a DVS instrument with a certain amount of sample. The mass at each relative humidity was recorded during a cycle of 0% RH-95% RH-0% RH at 25° C., and XRPD was collected before and after DVS test. The DVS adsorption curve of Form CSI is shown in FIG. 11. The result shows that the weight gain of Form CSI under 80% RH is 0.05%, and Form CSI is almost non hygroscopic.

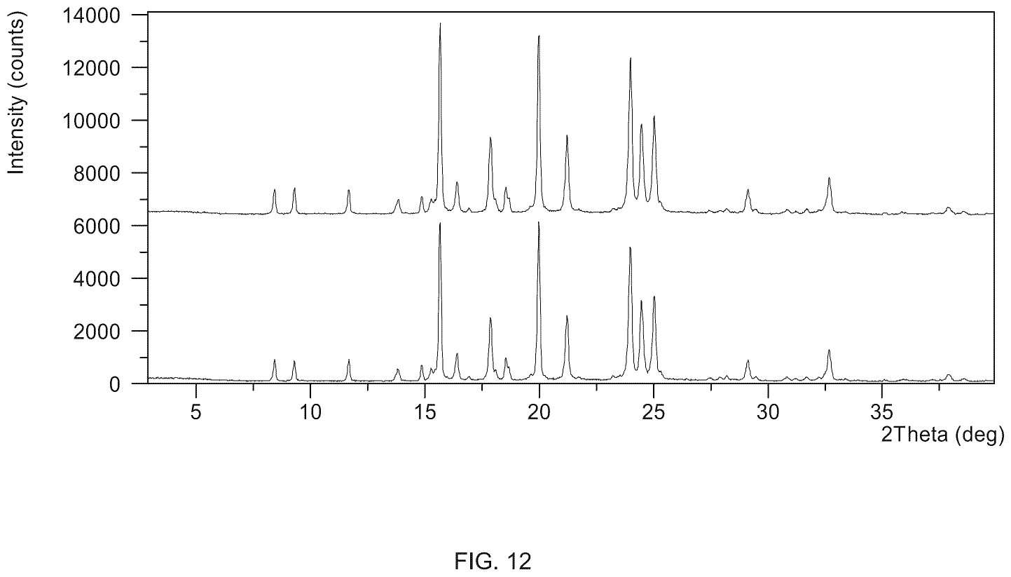

The XRPD overlay before and after DVS test is shown in FIG. 12. No form change is observed after DVS test of Form CSI, indicating that Form CSI has good humidity stability.

### Example 9 Physical Stability of Form CSI Upon Mechanical Force

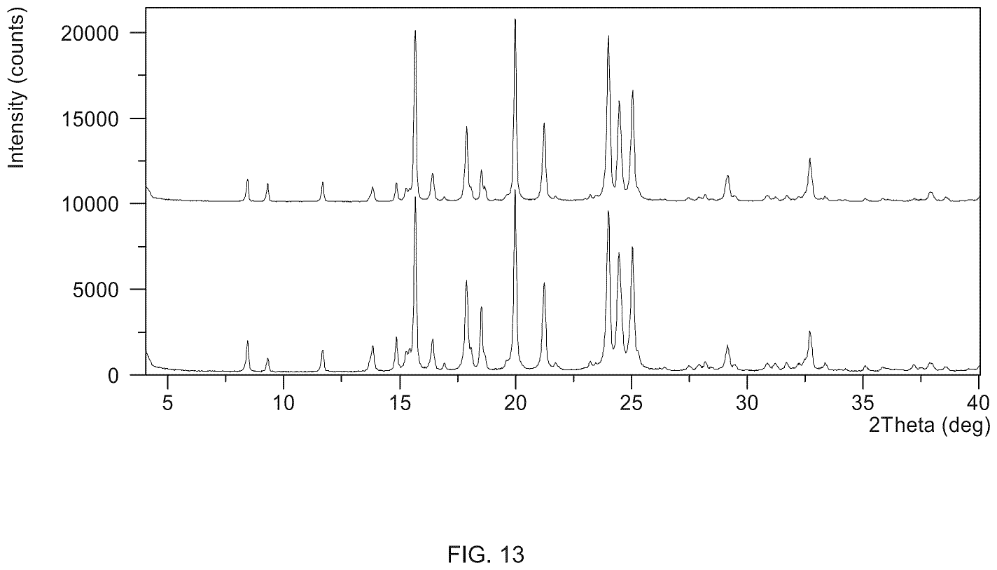

Form CSI was ground using a ball-milling at a vibration speed of 500 rpm for 5 minutes. XRPD was tested before and after grinding, as shown in FIG. 13. The results show that no form change was observed of Form CSI after grinding, indicating that Form CSI has good physical stability under mechanical force.

### Example 10: Stability of Form CSII

A certain amount of Form CSII sample was packaged with corresponding package conditions, and then stored at 25° C./60% RH and 40° C./75% RH. Crystalline form was checked by XRPD. The results are shown in Table 7. Form CSII kept stable for at least 1 month for both open and sealed (with desiccants) package conditions at 25° C./60% RH and 40° C./75% RH. The results show that the Form CSII has good stability and is more stable than the prior art solid.

TABLE 7

| Initial | | | Storage | |
|---|---|---|---|---|
| solid form | Storage condition | Package condition | time | Solid form |
| Form | Initial | — | — | Form CSII |
| CSII | 25° C./60% RH | Sealed (with | 1 month | Form CSII |
| | | desiccants) | | |
| | 40° C./75% RH | Sealed (with | 1 month | Form CSII |
| | | desiccants) | | |

### Example 11 Hygroscopicity of Form CSII

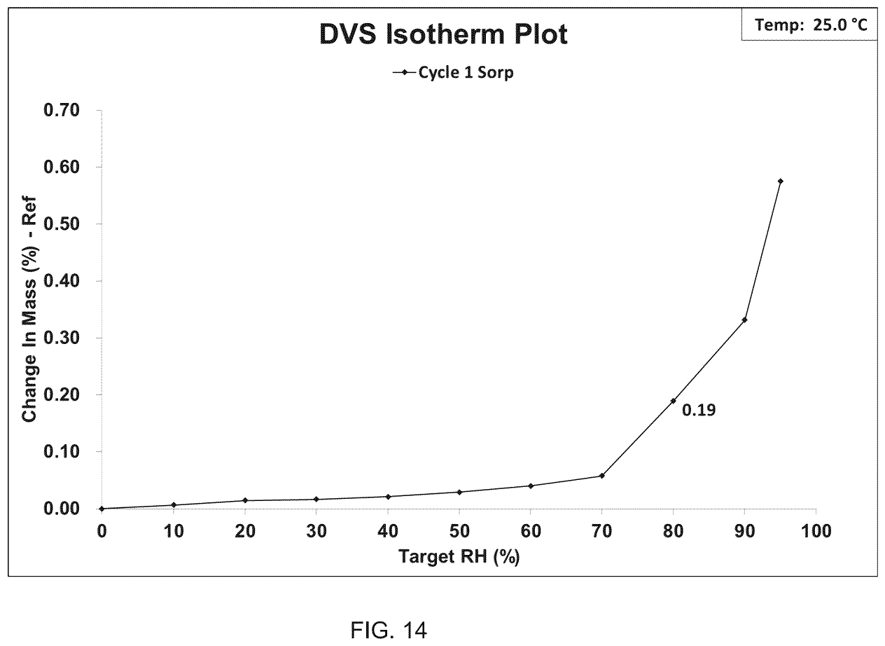

The hygroscopicity of Form CSII was determined by DVS instrument with a certain amount of sample. The mass at each relative humidity was recorded during the cycle of 0% RH-95% RH-0% RH at 25° C., and XRPD was tested before and after DVS. The DVS absorption curve of Form CSII is shown in FIG. 14. The result shows that the water uptake of Form CSII under 80% RH is 0.19%, and Form CSII almost non hygroscopic.

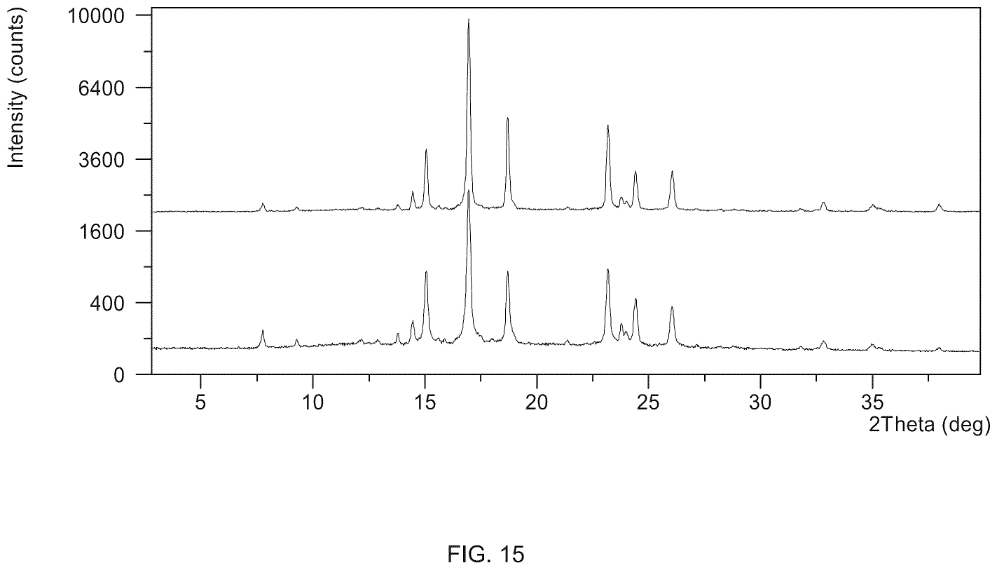

The XRPD overlay before and after DVS testing is shown in FIG. 15. No form change is observed after DVS test for Form CSII, indicating that Form CSII has good humidity stability.

### Example 12 Physical Stability of Form CSII Upon Mechanical Force

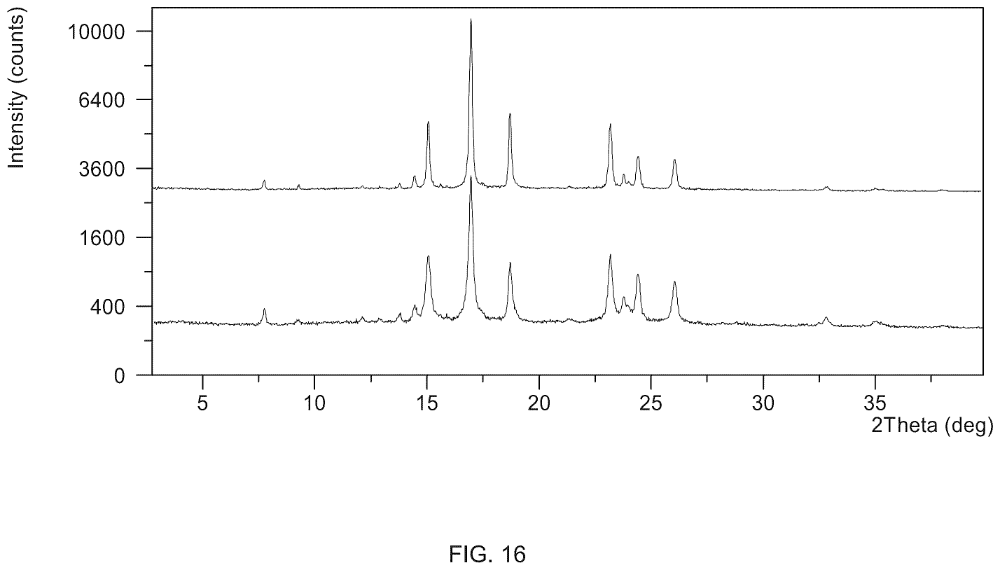

Form CSII was ground using a ball-milling at a vibration speed of 500 rpm for 5 minutes. XRPD was tested before and after grinding, and the results are shown in FIG. 16. The results show that no form change was observed after grinding, indicating that Form CSII has good physical stability under mechanical force.

The examples described above are only for illustrating the technical concepts and features of the present disclosure, and intended to make those skilled in the art being able to understand the present disclosure and thereby implement it, and should not be concluded to limit the protective scope of this disclosure. Any equivalent variations or modifications according to the spirit of the present disclosure should be covered by the protective scope of the present disclosure.

1-5. (canceled)

6. A crystalline form of Compound I 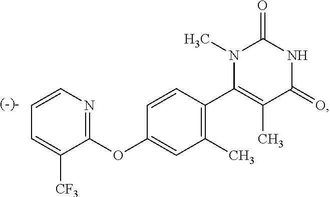 [SMILES: Cc1cc(Oc2ncccc2C(F)(F)F)ccc1-c1c(C)c(=O)[nH]c(=O)n1C]
wherein the X-ray powder diffraction pattern comprises characteristic peaks at 2theta values of 7.8°±0.2°, 15.1°±0.2° and 17.0°±0.2° using Cu-Kα radiation.

7. The crystalline form according to claim 6, wherein the X-ray powder diffraction pattern comprises at least one characteristic peak at 2theta values of 18.7°±0.2°, 23.2°±0.2° and 26.0°±0.2° using Cu-Kα radiation.

8. The crystalline form according to claim 6, wherein the X-ray powder diffraction pattern comprises at least one characteristic peak at 2theta values of 23.8°±0.2° and 24.4°±0.2° using Cu-Kα radiation.

9. The crystalline form according to claim 7, wherein the X-ray powder diffraction pattern comprises at least one characteristic peak at 2theta values of 23.8°±0.2° and 24.4°±0.2° using Cu-Kα radiation.

10. The crystalline form according to claim 6, wherein the X-ray powder diffraction pattern is substantially as shown in FIG. 4.

11. A pharmaceutical composition, wherein said pharmaceutical composition comprises a therapeutically effective amount of the crystalline form according to claim 6, and pharmaceutically acceptable excipients.

12. A method of preparing drugs of dopamine D1/D5 receptor agonists, comprising using the crystalline form according to claim 6.

13. A method of preparing drugs for treating Parkinson's disease, comprising using the crystalline form according to claim 6.
